(19) United States
(12) Patent Application Publication
(10) Pub. No.: US 2002/0065511 A1
(43) Pub. Date: May 30, 2002

(54) Patient stabilization system

(76) Inventors: Ricardo Guimaraes, Belo Horizonte (BR); Rod Ross, Mission Viejo, CA (US)

Correspondence Address: IRELL & MANELLA LLP, 840 Newport Center Drive, Suite 400, Newport Beach, CA, 92660, US

(21) Appl. No.: 09/727,080

(22) Filed: Nov. 29, 2000

(51) Int. Cl. A61B 18/20

(52) U.S. Cl. 606/5

(57) ABSTRACT

A system for detecting a movement of a patient's head during an ophthalmic procedure. The system may include one or more transmitters that can be attached to the head of the patient. The transmitters can transmit signals that are detected by corresponding receivers. The receivers may be located on a laser assembly that directs a laser beam onto the cornea of the patient. The system may have an indicator that is activated when one or more signals are not received, indicating that the patient's head has moved outside a predetermined range.

### BACKGROUND OF THE INVENTION

1. Field of the Invention The present invention relates to a detection system to determine whether a patient's head moves during an ophthalmic procedure.

2. Background Information

There have been developed various techniques to correct the vision of a patient. For example, there is a medical procedure that varies the curvature of a cornea using a laser. This procedure is commonly referred to as Laser in situ Keratomileuris (LASIK).

A LASIK procedure is performed by initially cutting a flap in the cornea to expose the stroma layer of the eye. A laser beam is then directed onto the stroma to ablate corneal tissue. After ablation, the flap is placed back onto the stroma. The result is a variation in the refractive characteristics of the eye.

LASIK procedures have been used to correct astigmatisms in an eye. Correcting for astigmatism requires a very accurate placement of the laser beam onto the cornea. Any movement by the patient during the laser ablation step may result in an unsuccessful procedure. There have been developed optical pattern recognition equipment that detects movement of the cornea. The recognition equipment can activate an alarm if the cornea moves beyond a predetermined range. These systems are relatively expensive to produce and purchase thereby increasing the cost of a LASIK procedure. It would be desirable to provide a detection system that can detect the movement of a patient's head during a LASIK procedure that is relatively inexpensive to produce.

### BRIEF SUMMARY OF THE INVENTION

One embodiment of the present invention includes a system for detecting movement of a patient's head during an ophthalmic procedure. The system may include a transmitter that transmits a signal to a receiver. The transmitter can be attached to the patient's head. An indicator may be activated if the receiver does not receive the transmitted signal.

### BRIEF DESCRIPTION OF THE DRAWINGS

FIG. 1 is an illustration showing an embodiment of a system of the present invention;

FIG. 2 is an illustration showing a plurality of transmitters attached to a patient's head;

FIG. 3 is a schematic of the system.

### DETAILED DESCRIPTION OF THE PREFERRED EMBODIMENT

In general the present invention includes a system for detecting a movement of a patient's head during an ophthalmic procedure. The system may include one or more transmitters that can be attached to the head of the patient. The transmitters can transmit signals that are detected by corresponding receivers. The receivers may be located on a laser assembly that directs a laser beam onto the cornea of the patient. The system may have an indicator that is activated when one or more signals are not received, indicating that the patient's head has moved outside a predetermined range.

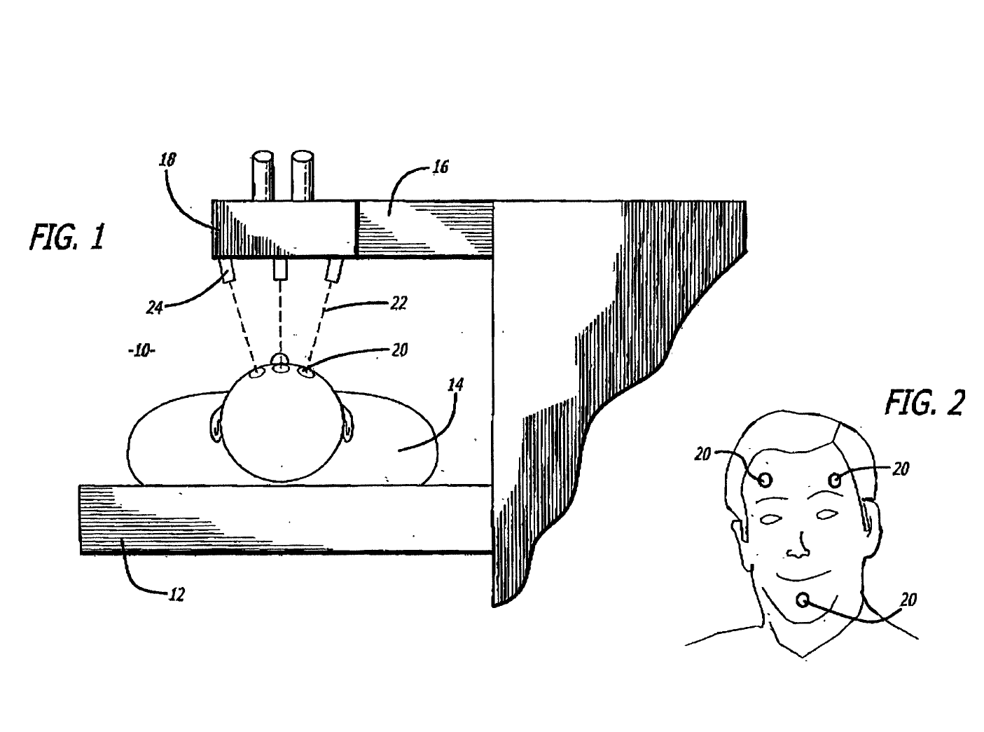

Referring to the drawings more particularly by reference numbers, FIG. 1 shows an embodiment of a system 10 of the present invention. The system 10 may include a support 12 that supports a patient 14. By way of example, the patient support 12 may be a table, although it is to be understood that the support 12 may be a chair or other support structure.

The patient 14 may be located beneath a laser assembly 16. The laser assembly 16 may include a laser 18 that directs a laser beam (not shown) onto a cornea (not shown) of the patient.

The system 10 may include a plurality of transmitters 20 that can be attached to the patient's head as shown in FIG. 2. The transmitters 20 may transmit signals 22 that are detected by a plurality of corresponding receivers 24 as shown in FIG. 1. The receivers 24 may be attached to the laser assembly 16.

The transmitters 20 may be magnets that emit magnetic fields. The receivers 24 may be magnetic transducers that convert magnetic fields into electrical signals. Although magnetic transmitters are shown and described, it is to be understood that the transmitters may emit different types of signals. For example, the transmitters 20 may each be a light source, such as a laser diode, or a light emitting diode that generate light beams detected by corresponding photodetectors. Alternatively, the transmitters 20 may actually be reflectors that reflect light emitted from lights sources, wherein the reflected light is detected by corresponding photodetectors.

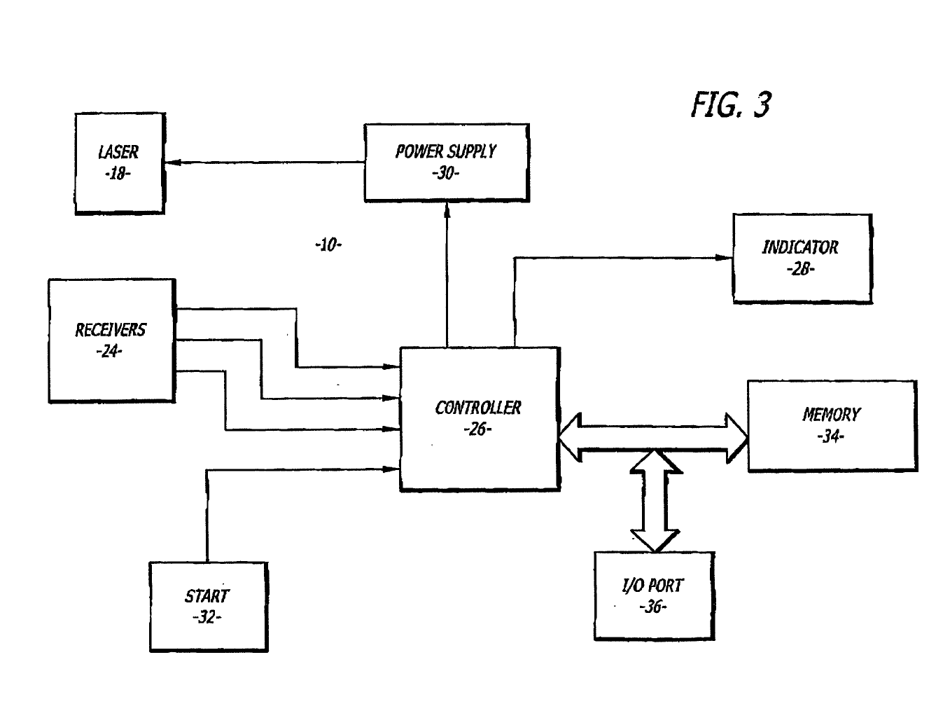

FIG. 3 shows an embodiment of the system 10, wherein the receivers 24 are connected to a controller 26 and an indicator 28. The controller 26 may be a microprocessor. The receivers 24 may provide input signals to the controller 26 in response to detection of the transmitted signals from the transmitters 20. The controller 26 may compare the input signals to a corresponding threshold value. The controller 26 may provide an output signal(s)/command if one or more of the inputs signals is less than the threshold value. The threshold value can be programmed to correspond to a range of motion by the patient's head.

The indicator 28 may be a light source that is illuminated when activated. Alternatively, or in addition to, the indicator 28 may include a speaker to emit a sound when activated. The controller 26 may include a speech program to generate a message to a surgeon when the input signals are less than the threshold value. Although a controller 26 is shown and described, it is to be understood other logic circuits may be used in the present invention. For example, the input signals may be provided to corresponding comparator circuits that have threshold signals provided as the other inputs to the circuits.

The controller 26 may be connected to a power supply 30 of the laser 18. The controller 26 can provide an output signal(s)/command to the power supply 30 to terminate power to the laser 18 when one or more of the input signals from the receivers 24 is less than the threshold value. The controller 26 can be programmed to deactivate the indicator 28 and reset the power supply 30 to allow generation of a laser beam when one or more of the input signals is equal to or exceeds the threshold value. The system 10 may further include a start button 32 that is connected to the controller 26. Depressing the start button 32 can initiate the controller 26 to begin comparing the input signals with the threshold value. The start button 32 can prevent the controller 26 from activating the indicator 28 when the system is not in use.

The system 10 may further have a memory device(s) 34 connected to the controller 26. The memory device 34 may include volatile and/or non-volatile memory. The controller 26 can perform a software routine in accordance with instructions and data stored in the memory device 34. Additionally, the memory device 34 may store and record when a patient moves their head during a procedure. The controller 26 and memory device 34 may be coupled to an input/output port 36 that is connected to a printer or other output device that allows an end user to review this data.

To perform an ophthalmic procedure in accordance with the present invention, the patient 14 is initially placed on the support 12 in a position so that the patient's cornea is properly aligned with the laser 18. The transmitters 20 are then attached to the patient's head and the start button 32 is depressed to activate the detection system. A corneal flap may be created in the cornea before the transmitters 20 are attached to the patient's head.

The laser 18 directs a laser beam (not shown) onto the cornea. If the patient moves their head beyond a predetermined range, the input signals from the receivers 24 will fall below the threshold value and the indicator 28 will be activated. The laser 18 may also be terminated to prevent ablation in an undesirable portion of the eye. The patient can move their head until the input signals equal or exceeds the threshold values, wherein the laser beam can be regenerated to complete the procedure. The transmitters 20 can be removed from the patient at the end of the procedure.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A system to perform an ophthalmic procedure on a cornea of a patient, comprising:
a first transmitter that can be attached to a head of the patient, said first transmitter transmits a first signal;
a first receiver that can receive the first signal from said first transmitter and provide an input signal; and,
an indicator that is activated when the first signal is less than a threshold value.

2. The system of claim 1, further comprising a controller that is connected to said receiver and said indicator and compares the input signal from said receiver with the threshold value and provides an output signal to said indicator if said input signal is less than the threshold value.

3. The system of claim 1, further comprising a second transmitter that can transmit a second signal to a second receiver, and a third transmitter that can transmit a third signal to a third receiver.

4. The system of claim 1, wherein said first transmitter transmits a magnetic signal.

5. The system of claim 1, further comprising a laser assembly that supports said first receiver.

6. The system of claim 1, wherein said laser assembly includes a laser that emits a laser beam onto the cornea, power to said laser is terminated when the input signal is less than the threshold value.

7. The system of claim 1, wherein said indicator can be illuminated.

8. The system of claim 1, wherein said indicator can generate a sound.

9. A system to perform an ophthalmic procedure on a cornea of a patient, comprising:
a support to support the patient;
a laser assembly that includes a laser which emits a laser beam onto the cornea;
a first transmitter that can be attached to a head of the patient, said first transmitter transmits a first signal;
a first receiver that is attached to said laser assembly and can receive the first signal from said first transmitter and generate an input signal; and,
an indicator that is activated when the input is less than a threshold value.

10. The system of claim 9, further comprising a controller that is connected to said receiver and said indicator and compares the input signal from said receiver with the threshold value and provides an output signal to said indicator if said input signal is less than the threshold value.

11. The system of claim 9, further comprising a second transmitter that can transmit a second signal to a second receiver, and a third transmitter that can transmit a third signal to a third receiver.

12. The system of claim 9, wherein said first transmitter transmits a magnetic signal.

13. The system of claim 9, wherein said laser assembly includes a laser that emits a laser beam onto the cornea, power to said laser is terminated when the input signal is less than a threshold value.

14. The system of claim 9, wherein said indicator can be illuminated.

15. The system of claim 9, wherein said indicator can generate a sound.

16. A method for performing an ophthalmic procedure, comprising:
attaching a first transmitter to a head of a patient, said first transmitter transmits a first signal;
receiving the first signal;
generating an input signal that corresponds to the first signal;
directing a laser beam onto a cornea of the patient; and,
activating an indicator if the input signal is less than a threshold value.

17. The method of claim 16, further comprising terminating the laser beam when the indicator is activated.
